(19) United States
(12) Patent Application Publication
(10) Pub. No.: US 2006/0253046 A1
(43) Pub. Date: Nov. 9, 2006

(54) Biopsy punch having limited length cutting edge

(76) Inventor: Gregory P. Wittenberg, Rapid City, SD (US)

Correspondence Address: SCHWEGMAN, LUNDBERG, WOESSNER & KLUTH, P.A., P.O. BOX 2938, MINNEAPOLIS, MN, 55402, US

(21) Appl. No.: 11/123,816

(22) Filed: May 6, 2005

(51) Int. Cl. A61B 10/00; A61B 17/32

(52) U.S. Cl. 600/567; 600/564; 606/184

(57) ABSTRACT

A skin biopsy punch has a limited length cutting edge, allowing a user to view a patient's skin through a top of the cutting edge and around a portion to be cut. In one embodiment, a portion of the handle adjacently coupled to the cutting edge is transparent. In a further embodiment, the cutting edge is supported by spaced apart connective sections coupled to a handle, allowing viewing of a skin lesion and the lesion's margins. By viewing the margins, the lesion may be cleanly removed or excised with a higher rate of negative or clear surgical margins.

### FIELD OF THE INVENTION

The present invention relates to biopsy punches, and in particular to a biopsy punch having a limited length cutting edge.

### BACKGROUND OF THE INVENTION

A skin biopsy punch is a device which samples a piece of skin. Biopsy punches have taken many different forms, with common punches having a cylindrical metal cutting blade that is circular or elliptical in shape. The blades may have a beveled cutting edge. Some punches are formed with an aperture or window positioned over the top of the cutting edge, allowing one to look down inside the blade to view tissue as it is being cut. However, these devices do not allow convenient simultaneous viewing of the tissue at or outside the cutting edge.

### SUMMARY OF THE INVENTION

A skin biopsy punch has a limited length cutting edge, allowing a user to view a patient's skin through a top of the cutting edge and around a portion to be cut. In one embodiment, a portion of the handle adjacently coupled to the cutting edge is transparent. In a further embodiment, the cutting edge is supported by spaced apart connective sections coupled to a handle, allowing viewing of a skin lesion and the lesion's margins. By viewing the margins, the lesion may be cleanly removed or excised with a higher rate of negative or clear surgical margins.

### BRIEF DESCRIPTION OF THE DRAWINGS

FIG. 1 is a cross section view of a biopsy punch according to an example embodiment of the invention.

FIG. 2 is a bottom view of the biopsy punch of FIG. 1 from a cutting edge end of the biopsy punch according to an example embodiment of the invention.

FIG. 3 is a partial cross section view an alternative biopsy punch according to an example embodiment of the invention.

FIG. 4 is a top view of an alternative biopsy punch according to an example embodiment of the invention.

FIG. 5 is a partial cross section view of a further alternative biopsy punch according to an example embodiment of the invention.

FIG. 6 is a bottom view of an alternative biopsy punch according to an example embodiment of the invention.

FIGS. 7A and 7B are perspective views of a biopsy punch having a limited length cutting edge according to an example embodiment.

FIG. 8 is a perspective view of an alternative biopsy punch having a limited length cutting edge according to an example embodiment.

FIG. 9 is a perspective view showing multiple connective sections of a view of a biopsy punch having a limited length cutting edge according to an example embodiment.

FIG. 10 is a perspective view showing multiple burs proximate a cutting edge of a biopsy punch according to an example embodiment.

### DETAILED DESCRIPTION OF THE INVENTION

In the following description, reference is made to the accompanying drawings that form a part hereof, and in which is shown by way of illustration specific embodiments in which the invention may be practiced. These embodiments are described in sufficient detail to enable those skilled in the art to practice the invention, and it is to be understood that other embodiments may be utilized and that structural changes may be made without departing from the scope of the present invention. Dimensions shown in the drawings may be exaggerated to more clearly show certain aspects, and such dimensions should not be taken as limiting of the scope of the invention. The following description is, therefore, not to be taken in a limited sense, and the scope of the present invention is defined by the appended claims.

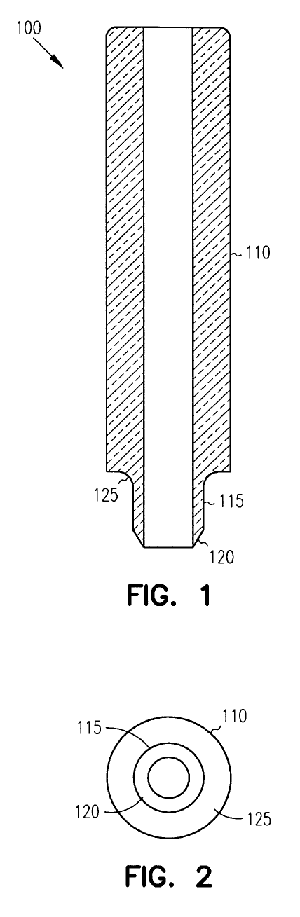

FIG. 1 shows a single piece biopsy punch generally at 100. Punch 100 has a handle 110 for gripping and manipulating the punch by hand or with other devices as desired. The handle 110 tapers into a cutting section 115 which is generally smaller in diameter than the handle 110 in some embodiments. The cutting section 115 tapers into a cutting edge indicated at 120. The cutting edge is sharp, to provide a means to take a biopsy of a skin lesion. Section 115 contains an exterior bevel on its distal end, ending in the cutting edge 120.

The cutting edge 120 may be used to sample a piece of skin or remove specific skin lesions in their entirety. The transparent nature of the punch or tool allows for visualization of the skin lesion including the lesion's margins. By viewing the margins, the lesion may be clearly removed or excised with a high rate of negative or clear surgical margins. A higher percentage of negative margins may result in better cure rates.

In one embodiment, the cutting section 115 and cutting edge 120 is formed of a material that is substantially clear, with little or no distortion. The material may be glass or plastic, such as a polycarbonate type material. The cutting edge 120 is sharp, beveled, and non-brittle in one embodiment to provide clean skin samples without debris. In one embodiment, the cutting edge 120 is as thin as possible to provide a sharp edge, yet strong enough to avoid chipping. The edge may be hardened using suitable radiation treatments in different embodiments.

The cutting edge may be sharpened, or formed in an injection mold. In one embodiment, the entire biopsy punch 100 is formed of a single piece of clear or transparent material. Section 115 and cutting edge 120 in one embodiment, is sufficiently long and sufficiently transparent to allow acceptable viewing of the area of skin to be cut, such as the lesion.

Other materials that may be used for the cutting edge 120, and cutting section 115 include polyvinyl chloride, styrene acrylonitrile, acrylonitrile styrene, unsaturated polyester, allylics, epoxies and vinyl esters. These plastics are rigid and translucent. Other materials may also be used, such as diamond, either mined or fabricated.

In one embodiment, the handle 110 is pencil shaped to be grasped easily between two or three fingers. A shelf 125 between the cutting edge and the handle 110 may be formed to prevent too deep a biopsy by providing a positive stop. The internal diameter of the cutting edge 120 may be any width desired, and is commonly between approximately 1 mm to 4 mms. While shown as circular in shape, the cutting edge may have different shapes, such as elliptical or even polygonal.

A bottom view of the biopsy punch 100 is shown in FIG. 2.

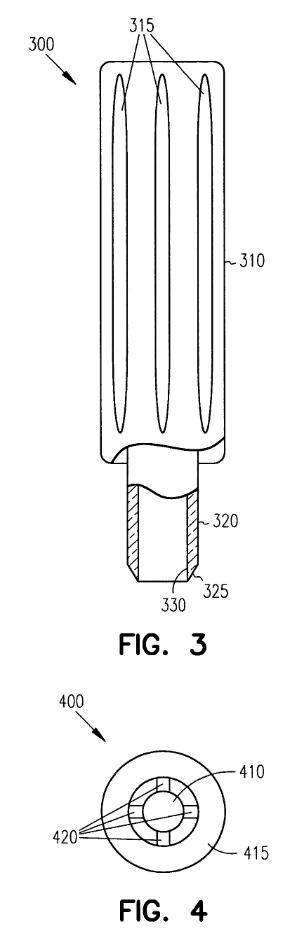

FIG. 3 shows an alternative biopsy punch 300 with a portion of it shown in cross section. Biopsy punch 300 comprises a handle 310 with gripping structures 315, such as ribs or grooves to facilitate gripping by a hand or other instrument. In this embodiment, the handle 310 is formed of a separate material from a cutting section 320, which is coaxially fixed at one end of the handle 310 such as by glue, heat, friction fit or other means. The handle may be transparent if desired, or made of any type of material that provides sufficient support for the cutting section, such as wood, plastic, metal, or the same material as the cutting section. The handle 310 may be formed in many different shapes, such as generally round, elliptical, triangular, polygonal, or any other ergonomic type of shape if desired. Cutting section 320 is beveled as indicated at 325, and ends in a sharp cutting edge 330.

FIG. 4 is a top view of yet a further alternative biopsy punch at 400. A metal cutting section 410 is coupled to a handle 415 by means of supports 420. In one embodiment, the metal cutting section is formed of stainless steel. The cutting section 410 may also be formed of a hardened plastic or other material that can hold a sufficiently sharp cutting edge in further embodiments. Four supports 420 are shown in this embodiment, but other numbers of supports may be utilized as desired. The supports may be glued or other wise adhered to support the cutting section 410 in a desired relationship to the handle 415.

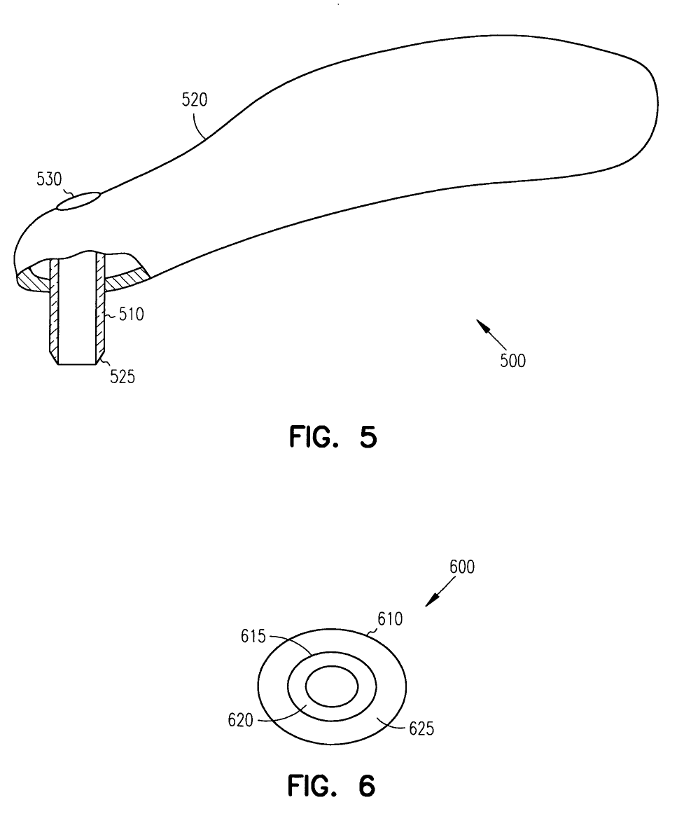

FIG. 5 is a partial cross section view of a further alternative biopsy punch 500 according to an example embodiment of the invention. In this embodiment, a transparent cutting section 510 is supported by a handle 520 that extends laterally away from the cutting section at an angle. Handle 520 in one embodiment, is shaped to fit in a hand, and the angle may be varied as desired to provide adequate usability for taking skin samples from various positions on a body. The shape of the handle may be varied as a function of desired ergonomics, and may come in different sizes for different sized hands. Tapered handles are used in one embodiment.

In one embodiment, the cutting section 510 has a first biopsy cutting edge 525 which may be formed of metal or hardened poly, and a second end 530 extending through the handle 520, allowing viewing of the skin or lesion being sampled through the second end, and also through the transparent cutting section 510. The handle may also be transparent, opaque, or partially transparent about the cutting section 510 to enhance viewability.

FIG. 6 is a bottom representation of the shape of an alternative biopsy punch 600. Punch 600 has a handle 610 for gripping and manipulating the punch by hand or with other devices as desired. In one embodiment, the handle is approximately 9 cm in length, providing a sufficient length for easy manipulation of the cutting edge or section without being too cumbersome. The handle 610 transitions into a cutting section 615 which is generally smaller in diameter than the handle 610 and is elliptical in shape. In some embodiments, other shapes may be utilized, such as polygonal shapes that may be designed for specific types of desired incisions. The cutting section 615 tapers into a cutting edge indicated at 620. The cutting edge is sharp, to provide a means to take a biopsy of a skin lesion. Metal cutting section 615 contains an exterior bevel on its distal end, ending in the cutting edge 620.

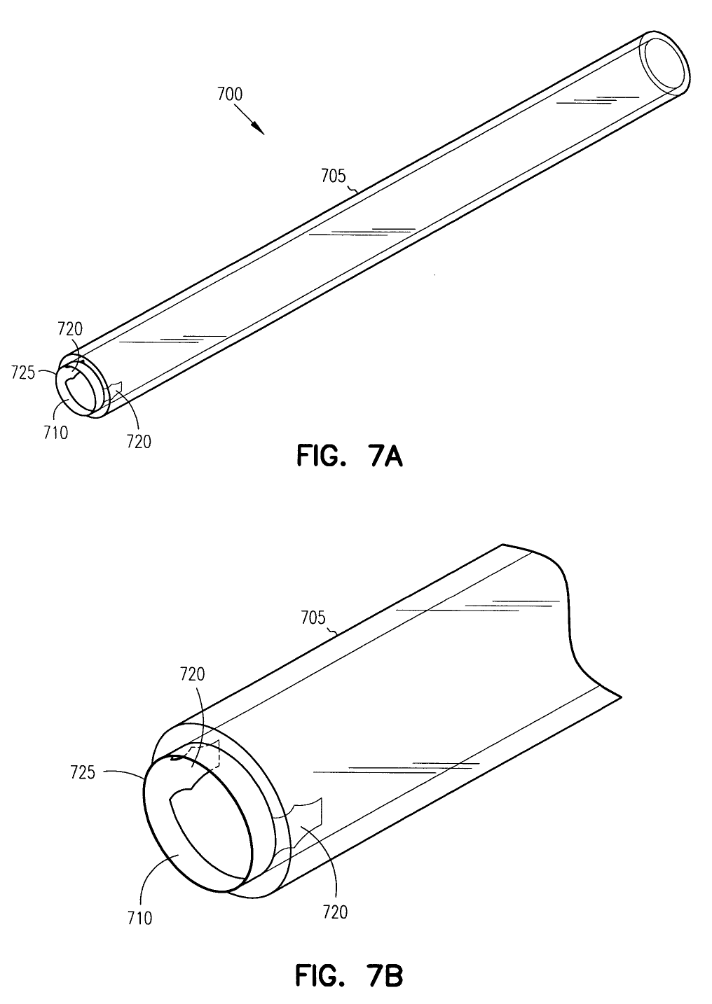

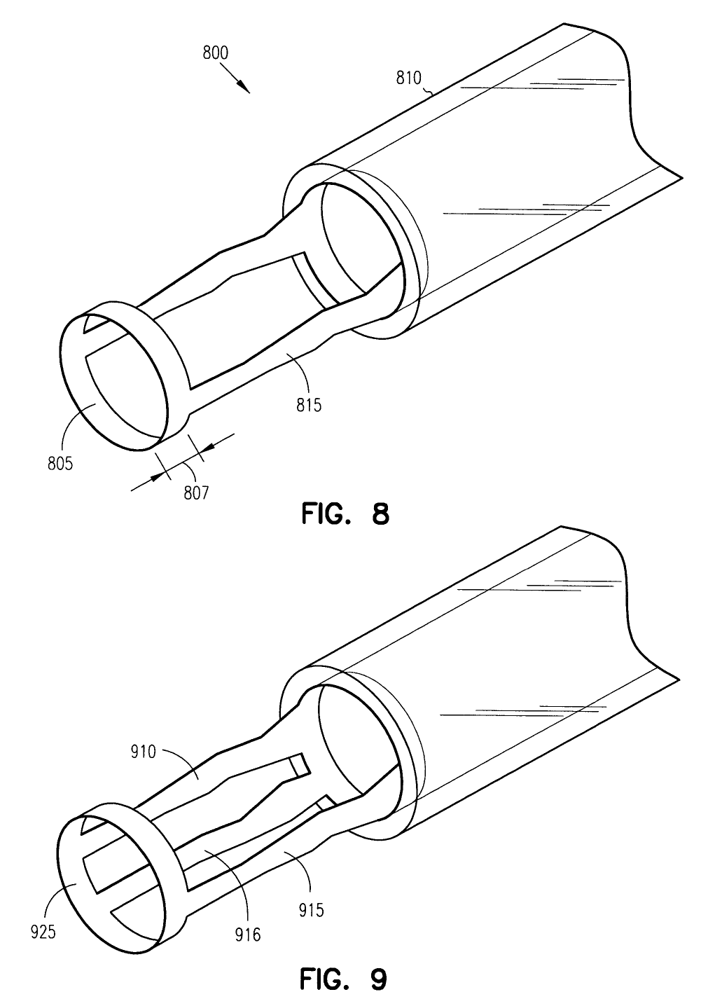

FIG. 7A shows a further embodiment of a biopsy punch at 700. Biopsy punch 700 has a clear handle portion 705 formed of a clear material such as polycarbonate, and a cutting section 710 formed of a different material, such as stainless steel or other metal. Ceramics or other materials capable of holding a surgically sharp edge may also be used. Cutting section 710 in one embodiment is fairly circular in shape, and has a limited length sufficient for taking a skin sample. Lengths from 1 mm to 4 mm may be used and still allow for convenient viewing of the skin sample being taken. Other lengths that provide sufficient cutting depth, yet visibility of the sample during use may also be used, such as lengths greater than 5 mm. Other shapes, such as elliptical or polygonal may also be used.

The cutting section 710 may be formed with tabs 720 extending towards the handle portion 705, allowing adjacent attachment of the cutting section to the clear portion of the handle. The cutting edge with tabs may be formed in a flat sheet, stamped out, and rolled to a desired shape. Other methods of making the cutting edge may also be used. In one embodiment, the clear handle portion is formed around the tabs 720 and partially encapsulates a portion 725 of the cutting section 710, while still providing sufficient length of cutting section for taking selected skin or lesion biopsies. This may be referred to as an integrated cutting section, shown in further detail in FIG. 7B.

The clear handle portion may be tapered toward the cutting section 710 to provide a smooth appearance and provide easier access and mobility proximate the skin. A hub may be located a desired distance from the cutting section, such as approximately 5 mm from the cutting section. The number of tabs may be varied in further embodiments, while still providing sufficient visibility and cutting section stability.

The clear handle portion 705 in one embodiment may be formed of a polymer such a polycarbonate as described above that is sufficiently clear to allow visibility of the skin sample. Further portions of the handle may be made of different materials as desired, which need not be transparent or clear, yet may also be shaped to provide an ergonomic grip.

A still further embodiment of the biopsy punch is shown at 800 in FIG. 8. Biopsy punch 800 also has a metal cutting edge 805, and a length 807 conducive to taking a desired biopsy. Length 807 is a limited length to allow viewing of the desired area of skin while taking the biopsy. In one embodiment, length 807 is approximately 2 to 3 mm. In further embodiments, it may be less than 2 mm, or may be as long as approximately 4 to 5 mm. The diameter of the punch may be between approximately 1 to 20 mm, with 4 to 8 mm typical. The limited length may be longer with larger diameter punches, while smaller diameter punches may use shorter lengths to allow adequate viewing of the desired area of the skin. While shown as round, the opening formed by the cutting edge may also be elliptical or polygonal, taking many different potential shapes as desired.

Cutting edge 805 may be metal or other suitably rigid material, and it is attached to a handle 810 by means of one or more connective sections 815. The connective sections may also be made of the same material as the cutting edge, provide sufficient stability and strength for using the cutting edge, and also provide visibility of the desired skin sample during use. Stainless steel is one such material. However, it should be noted that even clear connective sections may be used if desired. In one embodiment, the connective sections may be bowed outward to provide even better viewing of the desired area of the skin, allowing better visualization in corners. Such bowing should not greatly impair the structural support provided by the connective sections.

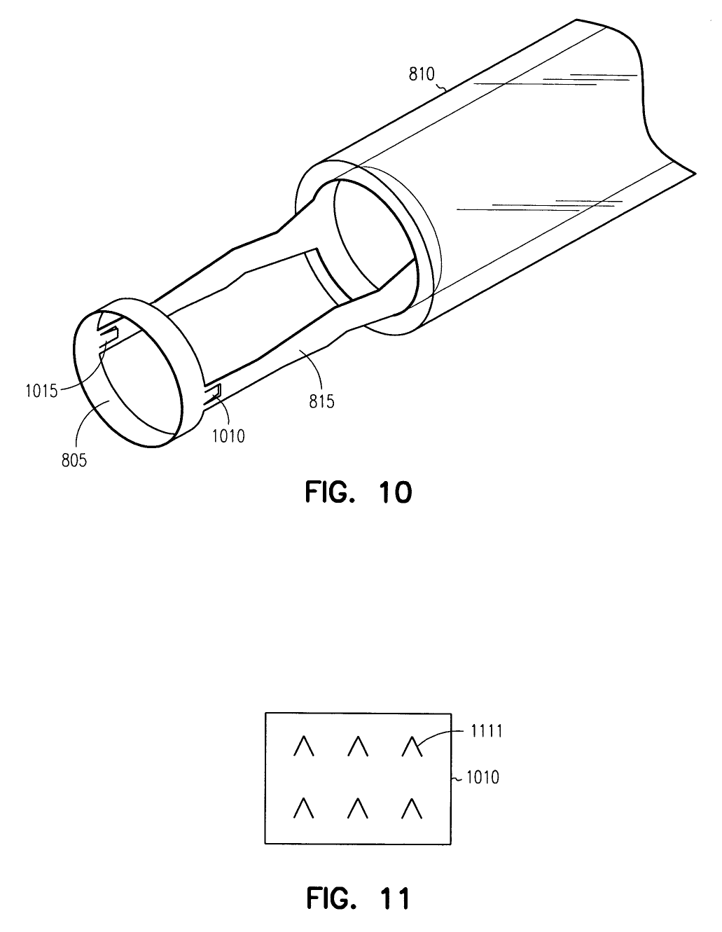

In one embodiment, two connective sections are provided as shown, on opposite sides of a longitudinal center of the punch 800. In further embodiments, three or more connective sections are provided. The connective sections may be equidistant from each other, or may be bunched somewhat, providing a larger opening for viewing as shown in FIG. 9, illustrating three connective sections at 910, 915 and 920. Such connective sections provide a larger opening indicated at 925 for providing sufficient viewing. Two smaller opening between the bunched connective sections are also a result.

In one embodiment, the connective sections may be provided with ridges or folds to provide increased strength. Many different techniques for providing structural integrity for sheet metal type supports are available. Other means of strengthening the connective sections may also be used as will be apparent to those skilled in the art. The connective sections in one example embodiment are between approximately 2 to 15 mm in length, with a width between approximately 1 to 7 mm.

In a further embodiment, a single connective section or support may be used with a window therein, such as a 180 degree or other angle window. In this embodiment, the length of the cutting edge may be limited only about the window, with the length opposite the window merging into the support itself.

FIG. 10 shows a biopsy tool with cutting edge 805 similar to that of FIG. 8. Additionally, one a plurality of burrs 1010 and 1015 are disposed about the cutting section, formed extending inward from the cutting edge 805. In one embodiment, the burrs are simply stamped from sheet metal used to form the cutting edge, and are angled slightly inward. The structure is such that the burrs act similar to fishing hook barbs, by grabbing the lesion during use, such that when the tool is pulled away from the skin, the lesion is also pulled upward by the barbs. This allows for easier removal of the lesion. As the lesion is lifted, a scalpel may also be used to free the underside of the lesion. The windows or opening between the support structures 815 allow easy access for removal of the lesion from the cutting edge.

The burrs maybe formed in many different ways, and may also be formed in different shapes. Substantially flat, punched out burrs 1010 and 1015 may be approximately 2 mm wide and 3 mm long in some embodiments. They may be aligned and formed out of the support structures as shown in FIG. 10, or may also be formed to the side of the support structures, extending inward from the cutting edge 805. More than two burrs may be used, and the sizes may be smaller or larger. The size may be dependent on the diameter of the cutting edge, and the length of the cutting edge. The size may also be varied depending on the depth of biopsy to be performed.

When the burrs are formed in the support structures, the support structures should be sufficiently wide to provide adequate support for the cutting edge. The structures should also not be so wide as to significantly decrease the ability to see the skin.

While the burrs are shown as substantially flat, they may be simply angled inward, or curved inward if desired. The surface of the burrs may also have smaller stamped inward portions, such as triangles 1111, or rectangular shapes pointing toward the handle, to provide an even more barbed type of function in retaining the lesion, as shown in FIG. 11. In one embodiment, the smaller stamped inward portions are formed in the support structures, or directly in the cutting edge, or extending from a top of the cutting edge, provided the cutting edge has a sufficient length, and the cutting functions of the cutting edge are not significantly impaired. Smaller stamped inward portions may be shaped and positioned to provide the ability to retain the lesion and help lift it away from the skin as the punch too is withdrawn.

The Abstract is provided to comply with 37 C.F.R. §1.72(b) to allow the reader to quickly ascertain the nature and gist of the technical disclosure. The Abstract is submitted with the understanding that it will not be used to interpret or limit the scope or meaning of the claims.

1. A biopsy punch comprising:
a handle;
a support coupled to the handle and having an opening; and
a limited length cutting section supported by the support such that skin being sampled by the biopsy punch is visible through the opening in the support.

2. The biopsy punch of claim 1 wherein the cutting section is hollow, allowing viewing of the skin being sampled through the opening in the support.

3. The biopsy punch of claim 1 wherein the cutting section has a chamfer on the cutting end that provides a sharp cutting edge.

4. The biopsy punch of claim 3 wherein the limited length of the cutting section is between approximately 1 mm and 4 mm.

5. The biopsy punch of claim 3 wherein the limited length of the cutting section is approximately 2 mm.

6. The biopsy punch of claim 1 wherein the support comprises two substantially diametrically opposed connective sections with openings therebetween.

7. The biopsy punch of claim 1 wherein the support comprises three spaced apart connective sections with openings therebetween.

8. The biopsy punch of claim 7 wherein the spaced apart connective sections are equally spaced.

9. The biopsy punch of claim 7 wherein the three spaced connective sections are bunched to provide one large opening and two smaller openings between the connective sections.

10. The biopsy punch of claim 1 wherein the cutting section is circular in shape with a diameter between approximately 1 and 20 mm.

11. A biopsy punch comprising:
a handle;
a plurality of connective sections coupled to the handle; and
a cutting section coupled to the connective sections, such that the connective sections are arranged to provide visibility into a center of the cutting section such that skin being sampled by the biopsy punch is visible.

12. The biopsy punch of claim 11 wherein the cutting section has a length of between approximately 1 mm to 4 mm.

13. The biopsy punch of claim 11 wherein the connective sections comprise two substantially diametrically opposed supports.

14. The biopsy punch of claim 11 wherein the connective sections comprise three spaced apart supports.

15. The biopsy punch of claim 11 wherein the connective sections are equally spaced.

16. The biopsy punch of claim 11 wherein the cutting section has a chamfer on the cutting end that provides a sharp cutting edge.

17. The biopsy punch of claim 11 wherein the connective sections comprise more than three spaced apart supports.

18. The biopsy punch of claim 11 wherein the cutting section is circular in shape with a diameter between approximately 1 and 20 mm.

19. A biopsy punch comprising:
a handle;
a cutting section having a limited length; and
means for coupling the cutting section to the handle to provide visibility into a center of the cutting section such that skin being sampled by the biopsy punch is visible.

20. The biopsy punch of claim 19 wherein the means for coupling comprises connective sections.

21. The biopsy punch of claim 19 wherein the means for coupling comprises a clear portion of the handle.

22. The biopsy punch of claim 21 wherein the means for coupling further comprises a plurality of tabs extending from the cutting section into the clear portion of the handle.

23. A method of taking a biopsy of skin, the method comprising:
placing a biopsy punch proximate an area of skin to be sampled;
viewing the skin to be sampled through a top portion of a limited length cutting section of the biopsy punch;
positioning the biopsy punch based on such viewing; and
taking a biopsy of the skin.

24. A biopsy punch comprising:
a handle;
a support coupled to the handle and having an opening; and
a cutting section supported by the support; and
a plurality of burrs disposed about the cutting section.

25. The biopsy punch of claim 24 wherein the burrs are formed in the support.

26. The biopsy punch of claim 24 wherein the burrs extend inward from the cutting section.
